(19) United States
(12) Patent Application Publication
(10) Pub. No.: US 2018/0021028 A1
(43) Pub. Date: Jan. 25, 2018

(54) FISTULA PLUG

(71) Applicants: Kaity Yu Emerson, East Brunswick, NJ (US); Chase Paul Monckton, Arlington Heights, IL (US); Adi Blanc, Cary, NC (US)

(72) Inventors: Kaity Yu Emerson, East Brunswick, NJ (US); Chase Paul Monckton, Arlington Heights, IL (US); Adi Blanc, Cary, NC (US)

(21) Appl. No.: 15/218,648

(22) Filed: Jul. 25, 2016

(51) Int. Cl. A61B 17/00

(52) U.S. Cl. CPC A61B 17/0057; A61B 2017/00628; A61B 2017/0061; A61B 2017/00641; A61B 2017/00659; A61B 2017/00663; A61B 2017/00862

(57) ABSTRACT

The present invention relates to a novel cap and plug device for occluding a fistula in a patient. This device has improved success rates over prior devices.

### COPYRIGHT NOTICE

A portion of the disclosure of this patent contains material that is subject to copyright protection. The copyright owner has no objection to the reproduction by anyone of the patent document or the patent disclosure as it appears in the Patent and Trademark Office patent files or records, but otherwise reserves all copyright rights whatsoever.

### BACKGROUND OF THE INVENTION

### Field of the Invention

The present invention relates to a medical device. In particular, it relates to a device and methods for occluding abnormal hollow structures or fistulae.

### Description of Related Art

A variety of abnormal passages, called fistula, can occur in the mammalian body. Fistulae are caused by a variety of conditions and circumstances. For example, fistulae can be caused by infection, congenital defects, inflammatory bowel diseases (such as Crohn's disease), irradiation, trauma, cancer, childbirth, and surgical procedures. Fistulae may occur in the circulatory, respiratory, digestive, genitourinary, and musculoskeletal systems. Examples include, but are not limited to, vesicovaginal, urethrovaginal, tracheoesophageal, gastrocutaneous, anorectal (anocutaneous), rectovaginal, rectovesical, recto-urethra and recto-prostatic fistulae. Common fistulae occur from the intestine to an opening in the skin.

Various methods and devices for repairing/occluding fistulae have been described. The exact procedure and/or device will depend on the type of fistula being treated. One technique for treating fistulae involves the use of a plug-like device, frequently there is a cap attached to the plug device to surgically attach the plug device to the patient.

The Cook® SIS Fistula Plug is manufactured from porcine small intestinal submucosa (SIS) and is intended for the repair of anal, rectal, and enterocutaneous fistulae. The modified SIS Fistula Plug (Cook® Surgisis™ RVP Recto-Vaginal Fistula Plug), also manufactured from porcine small intestinal submucosa (SIS), is supplied in a tapered configuration with a button to provide increased retention of the plug and improved blockage of the fistula.

The GORE® BIO-A® Fistula Plug is intended for use in anorectal fistulae. This plug device has a three-dimensional disk with tube mesh design and is comprised of the synthetic bioabsorbable material polyglycolic acid:trimethylene carbonate (PGA:TMC).

Unfortunately, while these devices with plugs do work, their failure rate is extremely high. The failure rate in use can be anywhere from 50% to 90%, with failure of the surgical attachment to the patient being the most likely cause of failure, rather than the plug itself. In addition, where the fistula is placed in a position where it is sat upon, e.g. for an anal fistula, they are extremely uncomfortable, causing the patient to move around and add to the complications of the device remaining in place.

### BRIEF SUMMARY OF THE INVENTION

The present invention relates to the discovery that attaching a novel cap to any of the known fistula plugs results in a fistula occlusion device with improved attachment properties when compared with previous devices, and a means of attaching a fistula plug in an occluding manner in a fistula tract. The present device also allows for flexibility within the body during use and has expansion/contraction properties as complies with motion around the fistula.

Accordingly, in one embodiment of the invention there is a device for occluding a fistula tract in a patient comprising:
a) a plug body designed for insertion into the fistula tract; and
b) a cap constructed of a medically acceptable material, attached to the plug body and designed to surgically attach the device to the patient, the cap comprising:
   i. a planar body portion attached to the plug body; and
   ii. an edge portion that is thicker than the planar body portion, the edge portion of a shape designed to conform to a suture placed through the planar body, wherein at least a portion of the suture wraps around the edge portion.

In another embodiment, there is a cap device for use in surgically attaching a medical device attached to the cap to a patient, the cap comprising:
a) a planar body portion attached to the plug body; and
b) an edge portion that is thicker than the planar body portion, the edge portion of a shape designed to conform to a suture placed through the planar body, wherein at least a portion of the suture wraps around the edge portion.

### BRIEF DESCRIPTION OF THE DRAWINGS

FIG. 1 is a top perspective view of a cap of the present invention sutured to a patient.

FIG. 2 is a side view cut through of a cap of the present invention.

FIG. 3 is a perspective of a cap attached to a fistula plug.

### DETAILED DESCRIPTION OF THE INVENTION

While this invention is susceptible to embodiment in many different forms, there is shown in the drawings, and will herein be described in detail, specific embodiments, with the understanding that the present disclosure of such embodiments is to be considered as an example of the principles and not intended to limit the invention to the specific embodiments shown and described. In the description below, like reference numerals are used to describe the same, similar, or corresponding parts in the several views of the drawings. This detailed description defines the meaning of the terms used herein and specifically describes embodiments in order for those skilled in the art to practice the invention.

### Definitions

The terms “about” and “essentially” mean±10 percent.

The terms “a” or “an”, as used herein, are defined as one or as more than one. The term “plurality”, as used herein, is defined as two or as more than two. The term “another”, as used herein, is defined as at least a second or more. The terms “including” and/or “having”, as used herein, are defined as comprising (i.e., open language). The term “coupled”, as used herein, is defined as connected, although not necessarily directly, and not necessarily mechanically.

The term “comprising” is not intended to limit inventions to only claiming the present invention with such comprising language. Any invention using the term comprising could be separated into one or more claims using “consisting” or “consisting of” claim language and is so intended.

References throughout this document to “one embodiment”, “certain embodiments”, and “an embodiment” or similar terms means that a particular feature, structure, or characteristic described in connection with the embodiment is included in at least one embodiment of the present invention. Thus, the appearances of such phrases in various places throughout this specification are not necessarily all referring to the same embodiment. Furthermore, the particular features, structures, or characteristics may be combined in any suitable manner in one or more embodiments without limitation.

The term “or” as used herein is to be interpreted as an inclusive or meaning any one or any combination. Therefore, “A, B or C” means any of the following: “A; B; C; A and B; A and C; B and C; A, B and C”. An exception to this definition will occur only when a combination of elements, functions, steps or acts are in some way inherently mutually exclusive.

The drawings featured in the figures are for the purpose of illustrating certain convenient embodiments of the present invention, and are not to be considered as limitation thereto. The term “means” preceding a present participle of an operation indicates a desired function for which there is one or more embodiments, i.e., one or more methods, devices, or apparatuses for achieving the desired function and that one skilled in the art could select from these or their equivalent in view of the disclosure herein and use of the term “means” is not intended to be limiting.

As used herein, the term “occluding a fistula tract” refers to a situation wherein a medical fistula, a tunnel or a tract, needs to be closed. The device for the present invention is designed to be utilized to have the plug portion be placed inside the tract of the fistula to allow for the healing process to close the fistula without foreign matter or infection entering the fistula tract. The cap is sutured over the wound opening (internal or external wound opening) to hold the plug in place. In one embodiment, only the cap is situated over the fistula opening and the cap closes or occludes the fistula.

As used herein, the term “patient” refers to an individual suffering from a fistula with an open fistula tract. The location can be any open location on the body but, in one embodiment, it refers to an anal fistula.

As used herein, the term “plug body” refers to the part of a fistula plug that is inserted into a patient's fistula tract to aid in the healing process of the fistula. Such plugs come in a known variety of styles and designs as described above and known in the art. The present invention's novel cap is attached to any fistula plug, as shown in the drawings, for the purpose of attaching the device to the patient.

As used herein, the term “cap” refers to the novel portion of the device of the present invention. It is a portion mounted essentially perpendicularly at the distal end of the plug body and is of a size larger than the fistula diameter so that it lays upon healthy tissue for the purpose of attachment to the patient's tissue. It comprises a planar body portion attached to the plug body; and an edge portion that is thicker than the planar body portion, the edge portion of a shape designed to conform to a suture placed through the planar body, wherein at least a portion of the suture wraps around the edge portion. An embodiment of the cap device is shown in the Figures. In one embodiment, the device is circular, but other shapes are readily obvious (such as oval or rhombus shapes) in view of the Figures and description of the device herein. The cap can be molded, in one embodiment, or made by any technique known in the art for a device of the description herein. Attachment to the plug is by any means known in the art, including molding, glue, sewing, and the like.

As used herein, the term “medically acceptable material” refers to polymeric materials for construction of the cap that are compatible with attachment to the tissue of a patient. Such polymeric materials are well known and one skilled in the art can easily choose an acceptable material. In one embodiment, the medically acceptable material is silicone, poly(lactic-co-glycolic acid) (PLGA), or poly(dimethyl siloxane) (PDMS).

As used herein, the term “surgically attach” and “suture” refers to the method of attaching the device to tissue for occluding a fistula tract in a patient. The plug portion is inserted into the fistula tract until the cap is resting against healthy patient tissue, such as healthy unbroken skin. Then the cap is sutured surgically to the patient's tissue, placing sutures through the planar portion around the edge portion and, finally, attaching the sutures to the tissue using known surgical techniques in view of the design and shape of the cap and in view of the Figures herein.

As used herein, the term “fiber reinforcement” refers to natural or synthetic fibers arranged and molded into the cap to reinforce the strength of the device. The fiber can be made of any surgically and medically compatible material such as poly(propylene), poly(amide) (Nylon), or poly(ester). In one embodiment, the fibers are arranged in a crosshatch pattern, however, any reinforcing pattern can be utilized, including randomized.

As used herein, the term “planar body portion” refers to the center portion of the device for occluding a fistula tract, which is essentially flat and either smooth or textured and/or designed to attached to the surrounding tissue. The proximal side has the plug body attached to it and the distal side does not need to have anything attached to it. The planar body portion can be fiber reinforced. In one embodiment, the planar body portion is from about 0.5 mm to about 1.5 mm thick. In one embodiment it is 1 mm thick.

As used herein, the term “edge portion” refers to the other circumferential edge of the cap portion, which is thicker than the planar body portion, as displayed in the Figures. This allows sutures to wrap around the edge portion of the cap through the planar portion wherein the thickness aids in preventing the sutures from tearing loose/through from the device. In one embodiment, it has a circular cross section, but other thickened shapes, such as an oval shape or the like, could also be utilized within the scope of a thickness aid. In one embodiment, the edge portion is fiber reinforced in the manner similar to the planar body portion. In one embodiment, the edge portion is from about 1 mm to about 3 mm thick. In one embodiment it is 2 mm thick.

### Drawings

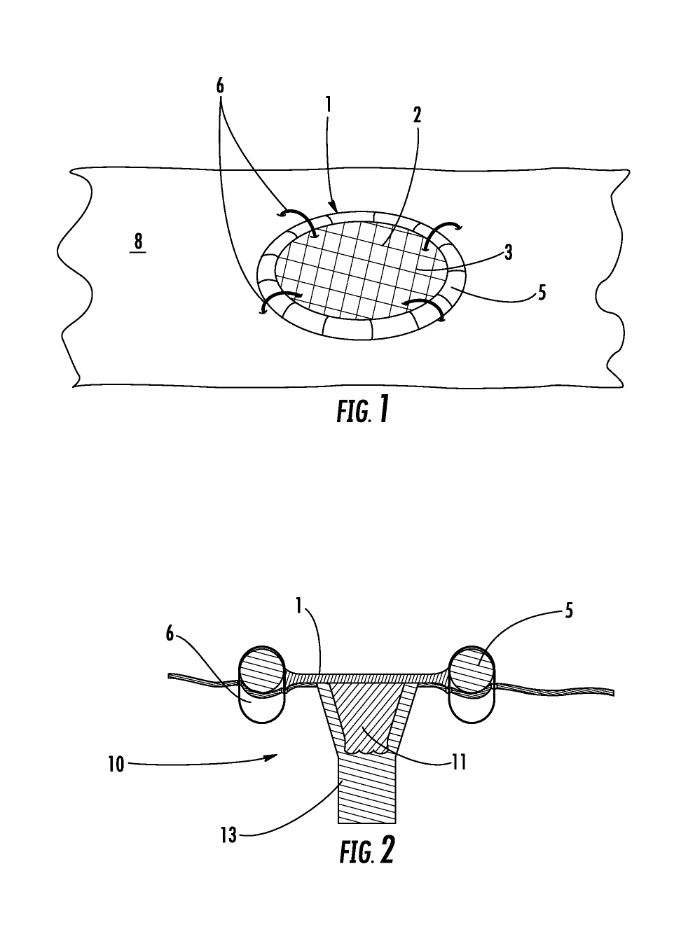

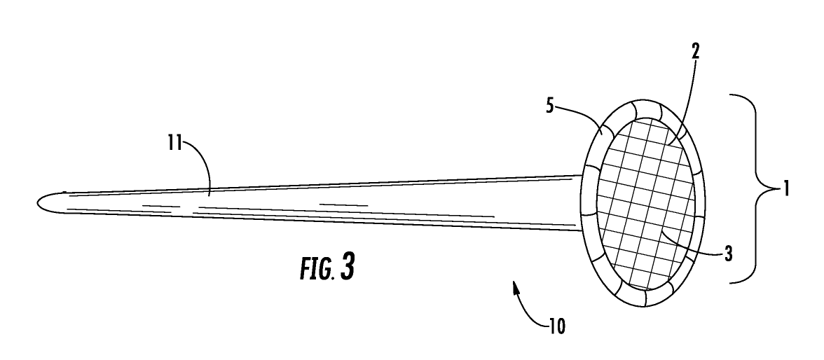

Now referring to the drawings, FIG. 1 is a top perspective view of the device of the present invention sutured onto a patient and the plug (not seen in this view) portion inserted into the fistula. Seen is cap 1, consisting of planar body portion 2 having fiber 3 embedded into the planar body 2. Cap 1 has an edge portion 5 which forms the circumference of the cap 1. It can be seen that while the planar body is flat, the edge portion 5 is cylindrical, having a circular cross section. The plug is not seen in this view, see FIG. 2. The cap is attached to a patient by putting sutures 6 through the planar body portion 2 (and fibers) over the edge portion 5 and ultimately attached to the tissues 8 of a patient.

FIG. 2 is a cross section of the entire device 10 for occluding a fistula. In this view, the cap 1 is seen sutured 6 to tissue 8. The plug portion 11 is shown partially as it is inserted into the fistula tract 13. FIG. 3 is a perspective of the device for occluding a fistula tract wherein the entire plug is seen and before insertion into fistula tract 13.

Those skilled in the art to which the present invention pertains may make modifications resulting in other embodiments employing principles of the present invention without departing from its spirit or characteristics, particularly upon considering the foregoing teachings. Accordingly, the described embodiments are to be considered in all respects only as illustrative, and not restrictive, and the scope of the present invention is, therefore, indicated by the appended claims rather than by the foregoing description or drawings. Consequently, while the present invention has been described with reference to particular embodiments, modifications of structure, sequence, materials and the like apparent to those skilled in the art still fall within the scope of the invention as claimed by the applicant.

What is claimed is:

1. A device for occluding a fistula tract in a patient comprising:
a) a plug body designed for insertion into the fistula tract; and
b) a cap constructed of a medically acceptable material, attached to the plug body and designed to surgically attach the device to the patient, the cap comprising:
a) a planar body portion attached to the plug body; and
b) an edge portion that is thicker than the planar body portion, the edge portion of a shape designed to conform to a suture placed through the planar body, wherein at least a portion of the suture wraps around the edge portion and is attached to the patient.

2. The device according to claim 1 wherein a cross section of the edge portion is circular.

3. The device according to claim 1 wherein the medically acceptable material is selected from the group consisting of silicone, poly(lactic-co-glycolic acid), and poly(dimethyl siloxane).

4. The device according to claim 1 which has a fiber reinforcement positioned in at least a portion of the device.

5. The device according to claim 4 wherein the fiber reinforcement is positioned in the planar body portion.

6. The device according to claim 4 wherein the fiber is constructed of a composition selected from the group consisting of poly(propylene), poly(amide), and poly(ester).

7. The device according to claim 1 wherein the cap is circular.

8. A cap device for use in surgically attaching a medical device attached to the cap to a patient, the cap comprising:
c) a planar body portion attached to the plug body; and
d) an edge portion that is thicker than the planar body portion, the edge portion of a shape designed to conform to a suture placed through the planar body, wherein at least a portion of the suture wraps around the edge portion.

9. The device according to claim 8 wherein a cross section of the edge portion is circular.

10. The device according to claim 8 wherein the medically acceptable material is silicone.

11. The device according to claim 8 which has a fiber reinforcement positioned in at least a portion of the device.

12. The device according to claim 11 wherein the fiber reinforcement is positioned in the planar body portion.

13. The device according to claim 11 wherein the fiber is constructed of a composition selected from the group consisting of poly(propylene), poly(amide), and poly(ester).

14. The device according to claim 8 wherein the cap is circular.
